(19) United States
(12) Patent Application Publication
(10) Pub. No.: US 2005/0074359 A1
(43) Pub. Date: Apr. 7, 2005

(54) Aircraft and passenger decontamination system

(75) Inventors: Eric W. Krieger, Mentor, OH (US); Thaddeus J. Mielnik, Concord, OH (US)

Correspondence Address: Thomas E. Kocovsky, Jr.;FAY, SHARPE, FAGAN, MINNICH & McKEE, LLP, Seventh Floor, 1100 Superior Avenue, Cleveland, OH, 44114-2518, US

(73) Assignee: STERIS INC.

(21) Appl. No.: 10/679,549

(22) Filed: Oct. 6, 2003

(51) Int. Cl. A61L 2/20

(52) U.S. Cl. 422/28; 422/33; 422/292; 422/300

(57) ABSTRACT

An integrated system for decontaminating an aircraft, its passengers, and items transported in the aircraft in the event of a known or suspected contamination of the aircraft by a pathogenic biological or chemical agent includes several mobile decontamination systems. A first decontamination vehicle (32) sprays a chemical decontaminant solution over an exterior of the aircraft. A mobile passenger recovery and treatment assembly (36) recovers passengers from the aircraft and decontaminates them with a human decontaminant solution. The assembly prevents escape of pathogenic agents into the surrounding environment. Luggage and other items are removed from the aircraft to a separate vehicle for decontamination or disposal. The aircraft interior (100) is then decontaminated with a gaseous decontaminant, such as hydrogen peroxide vapor delivered by a mobile generator (142).

### BACKGROUND OF THE INVENTION

The present invention relates to the decontamination of vehicles, such as those which carry passengers or cargo. It finds particular application in the decontamination of aircraft which are known or suspected of being contaminated with pathogenic microorganisms, such as anthrax, SARS, and the like. It will be appreciated, however, that the invention also finds application in the decontamination of passengers, cargo, and the interiors of a variety of vehicles, including cruise ships, trains, buses, and the like.

Commercial aircraft fly frequently between a large number of international destinations, posing the risk that the aircraft or people (passengers and crew) and cargo on board may become contaminated with pathogenic microorganisms, either through accidental or intentional means. Several pathogenic microorganisms, such as those responsible for Severe Acute Respiratory Syndrome (SARS) (a virus of the coronavirus type) and anthrax (Bacccilus anthracis) have been found to be capable of surviving on surfaces for several days or even months. Pathogenic microorganisms such as these may be introduced to the aircraft by infected passengers or carried in the cargo and luggage brought on board.

In addition to biological contamination, there is also the possibility for an aircraft to be intentionally contaminated with chemical contamination, such as chemical warfare agents.

For airlines to be financially viable, it is important that planes be returned to service as quickly as possible. In the case of aircraft contaminated or potentially contaminated with pathogenic microorganisms or chemical agents, there needs to be an assurance that the contamination has been removed before the aircraft is returned to service.

Microbial decontamination of rooms and buildings has been achieved in the past using chlorine dioxide gas. However, chlorine dioxide is highly toxic and must be recovered from the microbial decontamination process. Recovery of toxic gases from dilution air, leaking air, and the degassing of gas absorptive materials in the decontaminated room or building is difficult and time consuming. Further, care must be taken and monitors placed to ensure that the toxic gas does not escape into the surrounding areas.

Vaporized hydrogen peroxide has been used to microbially decontaminate small enclosures, such as sterilizers, and their contents. Hydrogen peroxide vapor systems provide dry, rapid, low-temperature decontamination of an enclosure that is contaminated with microorganisms, such as spore-forming bacteria. Keeping the temperature of the enclosure near room temperature eliminates the potential for thermal degradation of associated equipment and items to be sterilized within the enclosure. In addition, hydrogen peroxide readily decomposes to water and oxygen, which are not harmful to the operating technicians, people nearby, or people subsequently coming into contact with the treated space.

For optimally effective sterilization, the hydrogen peroxide is maintained in the vapor state. Sterilization efficiency is reduced by condensation. Typically, a solution of about 35% hydrogen peroxide in water is injected into a vaporizer as fine droplets or mist through injection nozzles. The droplets fall on a flat heated surface which heats the droplets to form the vapor, without breaking it down to water and oxygen. A carrier gas is circulated over the heat transfer surface to absorb the peroxide vapor.

One problem is the time taken to reduce the concentration of the hydrogen peroxide vapor in the enclosure to a safe level once sterilization is complete. In sterilizer enclosures, a vacuum is generally drawn to remove the vapor. Such a procedure would not generally feasible with large enclosures, such as aircraft, where even small leaks in the structure would prevent vacuum pressures of much below atmospheric pressure from being achieved.

The present invention provides a new and improved system and method of decontamination of aircraft and other vehicles which overcome the above-referenced problems and others.

### SUMMARY OF THE INVENTION

In accordance with one aspect of the present invention, a method for decontamination of an aircraft and contents thereof is provided. The method includes removing people from the aircraft. The people are decontaminated with a liquid decontaminant capable of reducing the pathogenic activity of at least one of a biological agent and a chemical agent, where present, on skin of the people. An interior of the aircraft is decontaminated with hydrogen peroxide vapor.

In accordance with another aspect of the present invention, a system for decontamination of a passenger aircraft and people on the aircraft is provided. The system includes an enclosure which receives people from the aircraft. Means for decontaminating the people are associated with the enclosure. A decontamination system is configured for selective connection with the aircraft for decontamination of an interior of the aircraft.

In accordance with another aspect of the present invention, a system is provided for decontamination of passengers from a vehicle or from within a facility who have been exposed to at least one of pathogenic biological agents and chemical agents. The system includes a mobile enclosure capable of being coupled to an exit door of the vehicle or facility for receiving passengers from the vehicle or facility. The enclosure spaces the received passengers from the surrounding environment. A decontaminant delivery system supplies a decontaminant to the enclosure. The decontaminant is capable of destroying the pathogenic biological or chemical agent. The delivery system includes at least one liquid outlet within the enclosure which sprays the decontaminant on the passengers.

One advantage of at least one embodiment of the present invention is that it provides an integrated system for decontamination of an aircraft and its passengers and luggage.

Another advantage of at least one embodiment the present invention is that it provides a mobile passenger decontamination system which is driven to a runway where a contaminated aircraft lands.

Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating a preferred embodiment and are not to be construed as limiting the invention.

FIG. 1 is a perspective view of an aircraft, passenger decontamination system, cargo recovery truck, and aircraft exterior decontamination truck according to the present invention;

FIG. 2 is a flowpath illustrating an aircraft, passenger, and cargo decontamination system according to the present invention;

FIG. 3 is a top plan view of the aircraft and the passenger decontamination system of FIG. 1 connected to a decontaminant generator;

FIG. 4 is a side sectional view of the passenger decontamination system of FIG. 3, coupled to an aircraft by a jet way assembly;

FIG. 5 is a schematic top view of the aircraft coupled to the passenger decontamination system of FIG. 1;

FIG. 6 is a schematic side view of a transport trailer raised into position adjacent a door of the aircraft of FIG. 1 by a scissor jack truck; and

FIG. 7 is a schematic view of the decontaminant generator of FIG. 3, coupled to inlet and outlet ports of the aircraft.

### DETAILED DESCRIPTION OF THE PREFERRED EMBODIMENTS

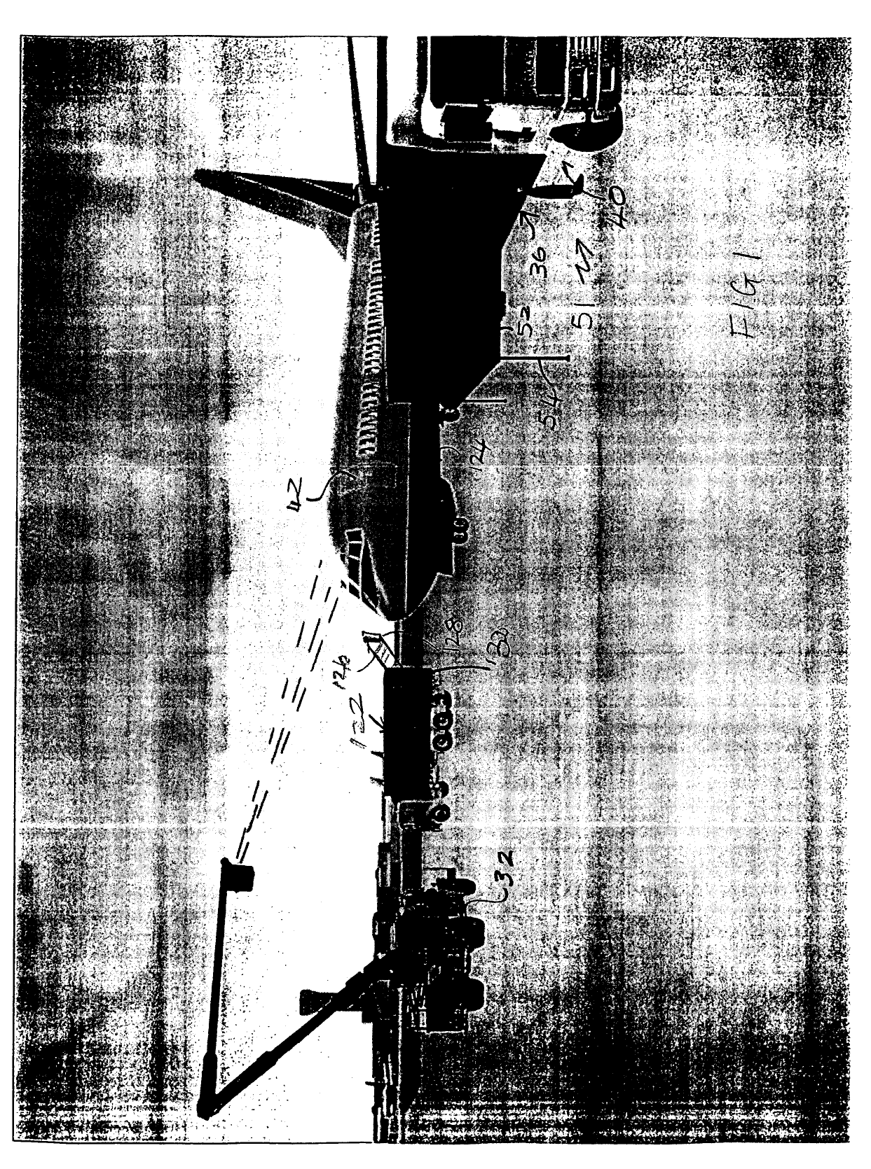

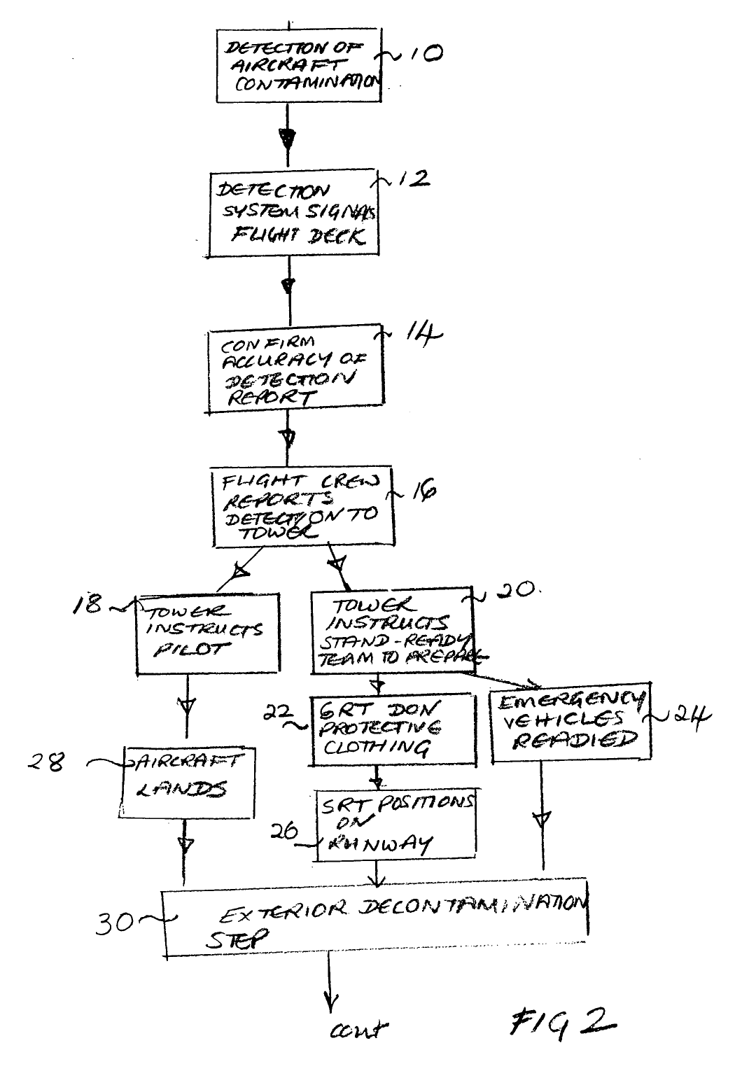

With reference to FIG. 1, a system for decontamination of an aircraft is shown positioned on a runway adjacent a contaminated aircraft. “Contamination,” as used herein refers to biological agents and/or chemical agents which are known to be pathogenic towards people or animals and/or are capable of causing severe illness. By “decontamination,” it is meant the destruction and/or removal of pathogenic agents (chemical and/or biological). In the case of biological pathogenic agents, the decontamination may rise to the level of sterilization, although lower levels of decontamination, such as disinfection and sanitization are also contemplated. In the case of chemical decontamination, conversion of chemical agents to non-harmful or less harmful species takes place. Preferably, the contamination is reduced to a level at which the pathogenic agents no longer pose a significant risk of pathogenic activity.

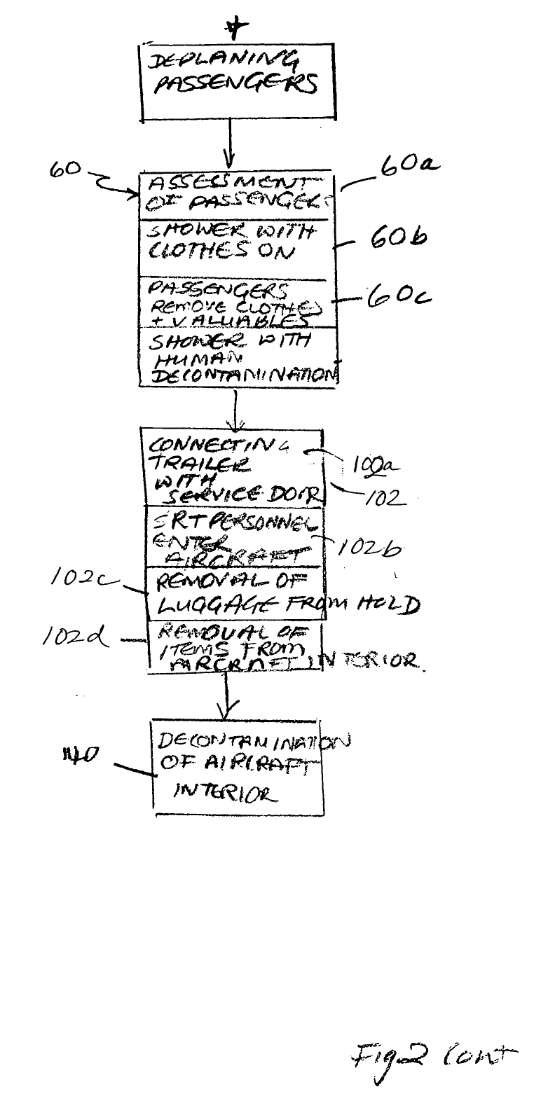

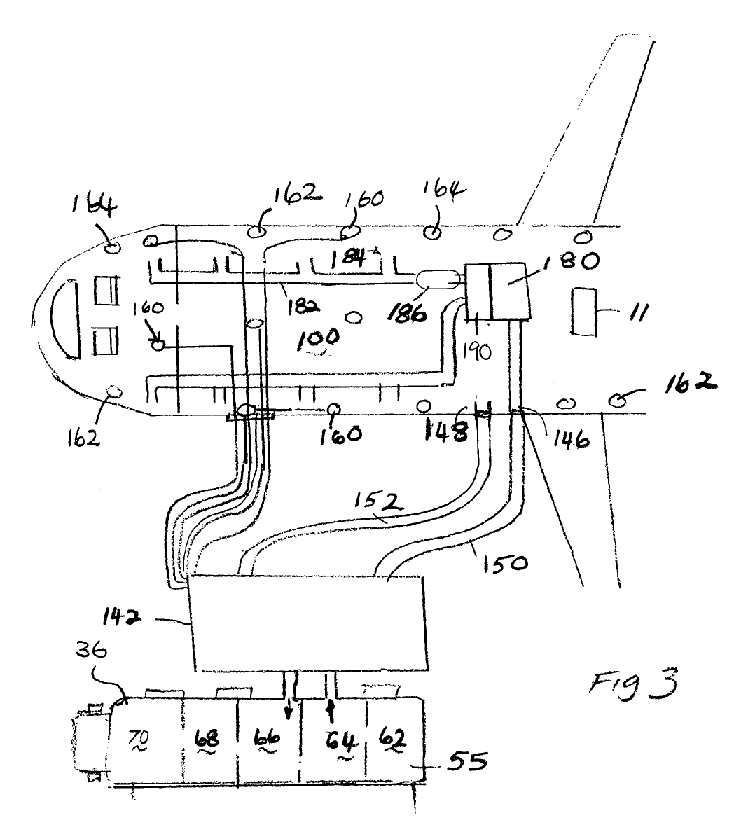

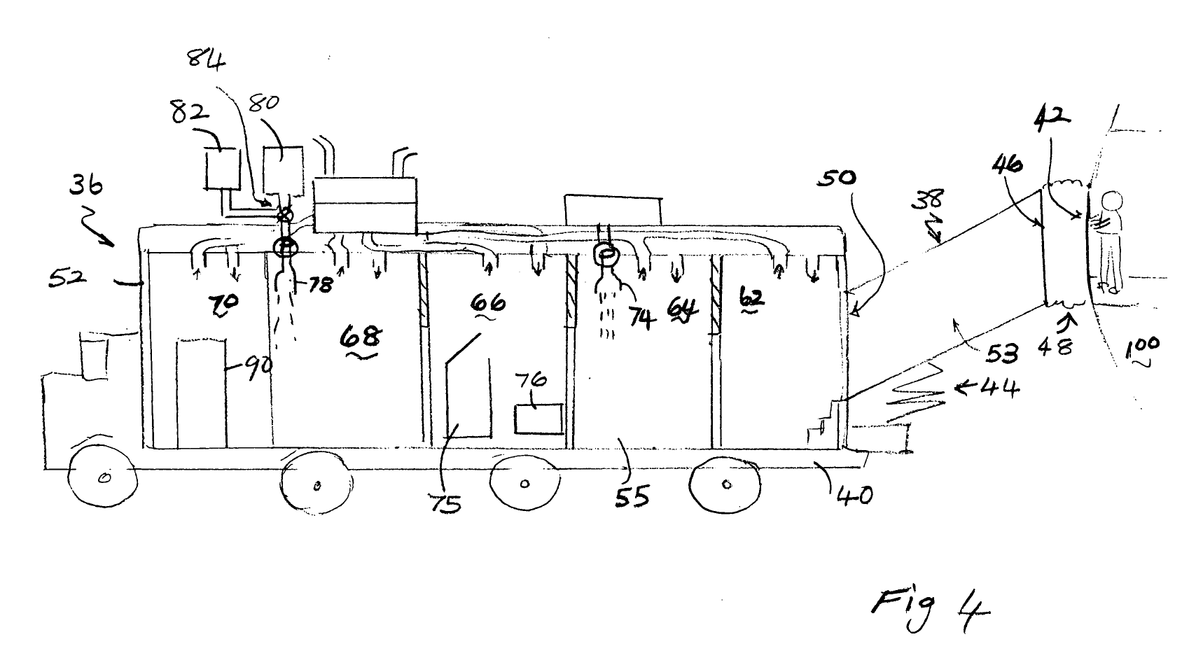

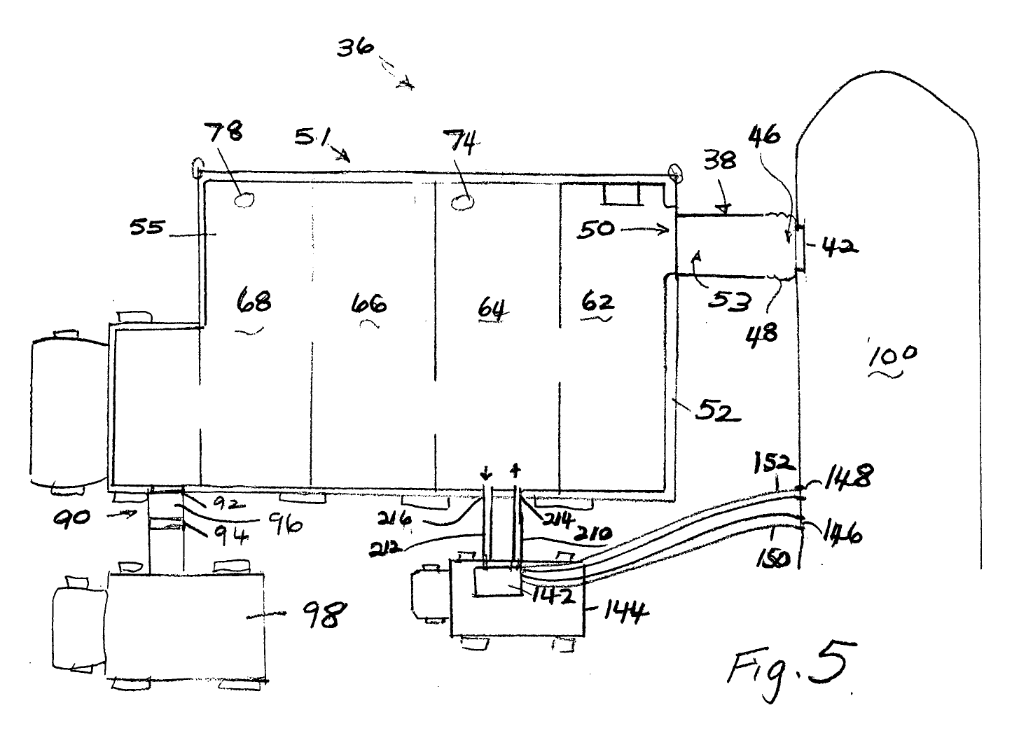

With reference to FIG. 2, a schematic diagram of an aircraft decontamination process is shown. The decontamination process includes several steps, some of which may be carried out concurrently or in an order other than that illustrated. It will be appreciated that fewer than all of the steps are optionally completed, depending on the type, level, and location of the contamination encountered. While particular reference is made to decontamination of passenger aircraft, it will be appreciated that the system is also applicable to the decontamination of cargo-carrying aircraft and other vehicles, such as boats, trains, road transportation vehicles, and the like.

In a first step 10, an actual or potential contamination of an aircraft is detected. Detection may be by a person, such as a flight attendant, pilot, or passenger, or by a detection system 11 within the aircraft (FIG. 3). The person or detection system 11 signals the flight deck that a chemical or biological agent has been detected in the aircraft (step 12). In step 14, the pilot or other member of the flight crew confirms the accuracy of the information. In step 16, a member of the flight crew radios the closest airport tower to alert ground staff that an actual or potential contamination has occurred. The tower then gives instructions to the pilot (step 18), which may include diverting the aircraft to a designated quarantine airport where a Stand-Ready Team (SRT) is located. The tower also alerts the SRT at the designated airport (step 20).

Once alerted, the SRT don personal protective equipment (PPE) (step 22). Emergency vehicles are also readied (step 24). These may include vehicles for transporting equipment for external and internal contamination of the aircraft and vehicles for aseptic removal of people on board and aseptic recovery of luggage and other cargo. The SRT is then positioned on the designated runway for arrival of the aircraft (step 26).

The aircraft lands at the designated quarantine airport and taxis to the area where the SRT has set up its equipment (step 28). Optionally, designated representatives of law enforcement are present to monitor activities, particularly if a terrorist threat or other criminal activity is suspected or known to be present.

A first step in the decontamination process is the external decontamination of the aircraft (step 30), although this may be carried out after people have safely deplaned. A mobile decontamination vehicle 32, filled with a decontamination solution effective against both biological and chemical agents, sprays the solution over the exterior of the aircraft, for example, by starting at the nose of the aircraft and working along the left side to the tail then along the right side, back to the nose. It is preferable to ensure complete coverage, although this may not be feasible in some circumstances. The external decontamination may be carried out with a truck conventionally used for applying a deicer to aircraft. Suitable decontamination solutions include, for example, solutions containing peracids, such as peracetic acid, hydrogen peroxide, hypochlorite, combinations thereof, and the like. One suitable decontaminant solution is DECON GREEN™, available from STERIS Corp., Mentor Ohio. Preferably, the decontamination solution is one which does not pose significant environmental hazards.

The exterior is confirmed clear of contamination based on specified parameters, such as the expected time taken for the decontamination solution to destroy potential pathogens under the ambient temperature. The step of deplaning the people in an aseptic manner then begins (step 34).

With reference to FIG. 4, a movable passenger recovery and treatment assembly 36 includes a jet way containment system 38, which is carried by a tractor-trailer 40, or other suitable vehicle, to a location adjacent an aircraft passenger exit doorway 42. The vehicle 40 is fitted with lifting equipment 44, which raises the jet way containment system 38 until an entry port 46 of the jet way containment system 38 is level with the doorway 42. A sealing system 48 seals around the aircraft door, prior to opening of the door. Passengers then exit the aircraft through the containment system 38 and pass through an entrance door 50 into a mobile passenger decontamination system 51 or a contained transport vehicle for transporting a stationary decontamination system. The passenger decontamination system in the mobile embodiment includes a trailer 52 which may be mounted on the tractor-trailer 40. It will be appreciated, however, that the trailer 52 and jet way containment system 38 may be mounted on separate vehicles or be a permanent or temporary static structure.

The jet way containment system 38 provides a passageway 53 between the aircraft door and the trailer entrance 50 though which the passengers and crew can be transferred to the enclosure in an aseptic manner—i.e., spaced from the external environment. This ensures that transfer of any biological or chemical contamination from the passengers or aircraft atmosphere to the external environment is eliminated or at least minimized.

In one embodiment, the trailer 52 expands such that it is partially supported on the runway on support legs 54 and partially on the trailer 40. The trailer defines an enclosure 55 which can be substantially or completely sealed from the surrounding environment. Injured or incapacitated passengers can be carried to the enclosure on a gurney or wheelchair.

Passenger decontamination (step 60) is at least partially carried out in the enclosure 55, depending on the type of contamination to which the passengers have been exposed. In the illustrated embodiment, the passengers proceed through a series of decontamination areas 62, 64, 66, 68, 70 within the decontamination system structure. Preferably, a plurality of such structures are provided with separate structures for male and female passengers.

The decontamination areas are arranged sequentially and are maintained under a negative pressure to minimize leakage of contaminants into the environment and cross contamination from one area to the next. For example, areas 62, 64, 66, 68, and 70 sequentially increase in pressure, with area 62 being at the lowest pressure. The negative pressure(s) may be achieved by a suitable negative pressure means 71, such as a heating, ventilating and air conditioning (HVAC) system, which preferably includes a filter system 72. Alternatively, a vacuum pump fitted with a filter system is used, or the like. The filter system filters air removed from the enclosure to remove microorganisms and chemical agents. To this end, the filter system may include a fine filtration medium, such as a HEPA filter, and/or a chemical filter medium, such as carbon. Inlet and outlet vents 73 are provided in each of the areas through which the contaminated air can be withdrawn and air filtered of contaminants returned. Optionally, fresh air is introduced to the enclosure from the exterior environment. Used air, which has passed through the filter may be released to the environment and/or recirculated to the enclosure. The negative pressure means optionally also creates a negative pressure on the jet way passageway 53.

In a first area 62, SRT medical personnel assess the passengers as to their medical condition (step 60a). In a second area 64, the passengers shower with clothes on (step 60b) of the human decontamination process). This initial shower is intended to reduce the contamination load before undressing. Optionally, this step is eliminated. A shower head 74 sprays a liquid over the passengers. Other methods of liquid contact are also contemplated, such as immersion in liquid. The shower liquid may consist of water. Optionally, the liquid includes a microbial decontaminant and/or surfactants and other chemicals to aid destruction or removal of contamination from the person and/or clothing. Suitable microbial decontaminants include solutions of a peracid, such as peracetic acid. The waste water is recovered and subsequently processed, as necessary and appropriate, to the identified contaminant.

In a third area 66, the passenger undress (step 60c). The clothing is placed in a sealable hazardous medical container 75 for disposal or more rigorous microbial decontamination. Preferably, jewelry, credit cards, and non-clothing items of value from the person are secured in an appropriate sealable container 76 with documented identification. The container 76 (and optionally container 75) is sealed and transported to a suitable decontamination site, which may be within the enclosure or remote therefrom. This step is preferably carried out after internal decontamination of the enclosure, described in greater detail below.

The passengers proceed to a fourth area 68, where they shower under a liquid outlet, such as a shower head 78 for a selected time using a human decontamination solution containing appropriate chemistry for the identified contamination (step 60d). Preferably, several shower heads, each one in a separate stall (not shown) are provided so that several passengers can shower concurrently. The showering time and chemistry are preferably selected to ensure decontamination of the passengers' skin to a level at which the chemical or biological agent can be expected to be removed and/or destroyed.

The chemistry is preferably one which is effective for destruction/inactivation of both biological and chemical agents, yet which is safe to utilize on the passenger. Antimicrobial agents which are safe for use on humans are suitable for this step, e.g., pre-surgery swabs.

Examples of suitable non-toxic decontaminants include peracids, such as peracetic acid, halogen solutions, halogen compounds and solutions of halogen compounds (e.g., sodium hypochlorite, chlorhexidine digluconate, cetyltrimethylammonium, -benzylammonium, and -diammonium halides, cetyltributyl-phosphonium halides, dodecyltrimethylammonium halides, tetradecyltrimethylammonium halides, and the like), phenolic compounds and halogenated phenolic compounds in solution (e.g., Triclosan), selenium sulfide, asiatic acid, benzoyl peroxide, minocyclin, and the like, alone or in combination.

A peracetic acid solution at a concentration of about 200 to about 4000 ppm, preferably, about 1000 to 2000 ppm is suitable for this step and is effective against a large number of chemical and biological pathogens. The chemistry may also include surfactants, such as conventional soaps, sequestrants, and other components designed to aid removal of the contamination.

The shower head 78 may be connected with a source of a human decontamination solution, such as a container (not shown). Alternatively, as illustrated in FIG. 4, the shower head is connected with a water source 80 and also with a source 82 of concentrated decontaminant. The water and decontaminant are mixed at a mixing valve 84 to form the decontaminant solution. The container of decontaminant solution or separate water and concentrated decontaminant may be carried on the trailer 52. Alternatively, one or more of these is connected with the truck by suitable hoses. Mains water may be used if an outlet is close by. Gravity feed or suitable pump(s) are used to deliver the decontaminant solution from the source(s) to the shower head.

Alternatively, the human decontaminant is packaged in single dose containers. After wetting with the shower, the passenger applies the decontaminant, using it all, and remains covered with it for a prescribed time before rinsing it off.

Following the shower, the passengers proceed to a fifth area 70 for drying off. They are issued standard clothing and, dependent on the medical condition of the passenger, security concerns, or the need to de-brief the passengers, they are directed to an appropriate recovery area, either within the enclosure, or elsewhere. Optionally, as shown in FIG. 5, an exit port 90, such as an interlock system with sequentially opening double doors 92, 94, connected by a passageway 96, allows the passengers to leave the trailer safely to an aseptic transport vehicle 98.

It will be appreciated that two or more passenger recovery and treatment assemblies 36 may be in operation concurrently, processing passengers from separate exit doors of the aircraft.

Once all the passengers have exited the aircraft, the aircraft door 42 is closed.

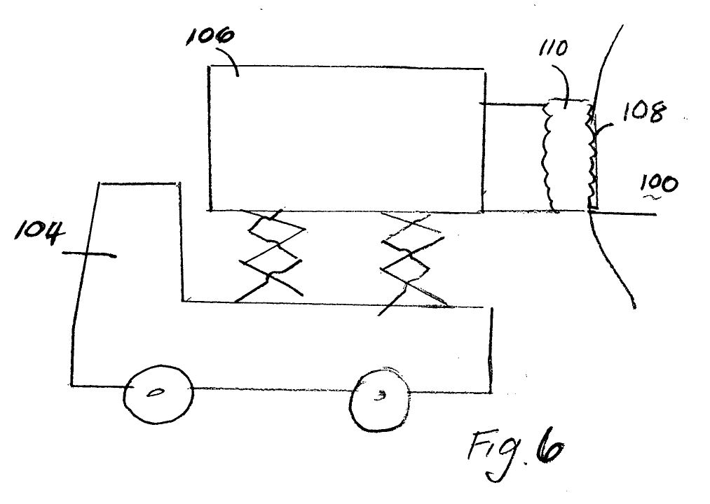

Following the aseptic processing of the passengers and the disposition of those passengers, the removal of contaminated/potentially contaminated materials from an interior 100 of the aircraft is initiated (step 102). It will be appreciated that this step may be commenced as soon as the last passenger or crew member has exited the plane. As shown in FIG. 6, a suitable vehicle, such as a scissor jack truck 104 raises a SRT transport trailer 106 to a service door 108 of the aircraft and a sealing member 110 creates an airtight seal around the door 108 (step 102a). SRT and law enforcement officials board the aircraft from the trailer 106 via the sealing member (step 102b). The sealing member 110 eliminates or substantially reduces the risk of cross-contamination or release of the contaminating agent into the atmosphere. The SRT and/or a law enforcement officer make a security sweep in full PPE to assess whether there may be items in the aircraft which pose a particular threat.

Concurrently, recovery of the luggage and other cargo from the belly of the aircraft is commenced (step 102c). A cargo recovery vehicle 122 (FIG. 1) is brought into the proximity of a cargo hold door 124. The truck 122 carries a jet way containment system 126 which includes a sealing member 128 similar to member 48 for forming an airtight seal around the cargo hold door. As with jet way containment system 38, the system 126 provides an enclosed passage for transfer of items without posing a risk of contaminating the exterior environment. Luggage is transported by an enclosed conveyor (not shown) via the jet way containment system 126 into an interior of the recovery truck. The recovered items are either disposed of, such as by incineration, and/or treated externally with vapor hydrogen peroxide or other suitable decontaminant and or a liquid decontaminant formulation, such as Spor-Klenz®, available from STERIS Corp., Mentor, Ohio. The recovered items can then be transported to a site where further processing is carried out. This further processing may include decontamination of the packaging and contents with a suitable gaseous decontaminant, such as ethylene oxide.

Within the aircraft, carry-on items are removed from the overhead compartments by the SRT personnel and aseptically moved into the same recovery truck 122 or a separate recovery truck (not shown) using a similar aseptic jet way system (step 102d). Other items on the aircraft which are considered to be expendable, such as magazines, blankets, pillows, and galley items, are treated as hazardous waste, and removed to a recovery truck for disposal.

Internal decontamination of the aircraft is then commenced (step 140) Decontamination is preferably carried out with a gaseous oxidizing agent, preferably hydrogen peroxide in vapor form. Hydrogen peroxide vapor has been shown to be effective against a variety of known biological and chemical pathogenic agents, such as hard to destroy spores of Bacillus stearothermophilus, Bacillus anthracis, smallpox virus, and the like. It is also effective at or close to room temperature (e.g., 15-30° C.), making it suitable for decontamination of aircraft interiors with little or no heating. Optionally, the aircraft's own heating system is used to heat the aircraft interior to a temperature which is effective for decontamination. Or, separate heaters (not shown) may be placed around the aircraft interior.

Hydrogen peroxide vapor has a good material compatibility, rendering it safe for use with a variety of equipment and materials, including electronic equipment, such as computers and other flight control equipment. It also degrades to water and oxygen over time. While the system is described with particular reference to decontamination with hydrogen peroxide vapor, it will be appreciated that other gaseous decontaminants are also contemplated, alone or in combination.

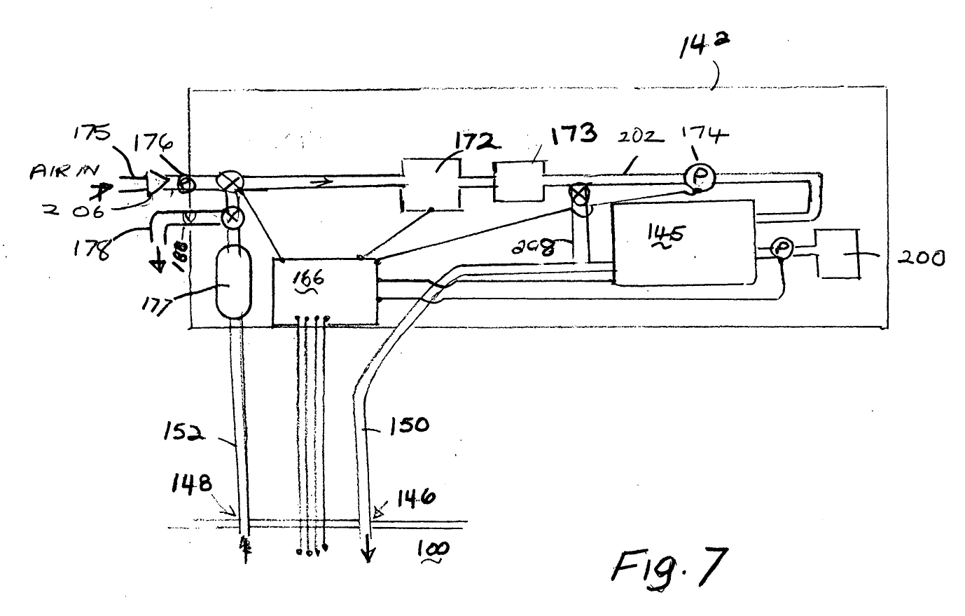

As shown in FIGS. 3, 5 and 7, a mobile decontaminant generation system 142 is transported on a suitable vehicle 144 to the aircraft. In the case of hydrogen peroxide as the decontaminant, the generation system preferably includes a vaporizer 145 (FIG. 7) for converting liquid hydrogen peroxide solution to vapor. A suitable hydrogen peroxide generation system 142 is a VHP® 1000 generator, available from STERIS Corp, Mentor, Ohio. Such systems are capable of sterilizing enclosed spaces of up to 200,000 cu. ft. For large aircraft interiors, two or more of such vaporizers may be employed. The aircraft has suitable fuselage inlet and outlet connection ports 146, 148, to which inlet and outlet hoses 150, 152 are connected, respectively for connecting the aircraft ventilation system and interior with the vapor generation system (step 140a).

Concurrently, the SRT inside the aircraft place one or more of hydrogen peroxide monitoring probes 160, biological indicators 162, and chemical indicators 164 (FIG. 3) at suitable locations around the interior 100 of the aircraft (step 140b). The indicators 162, 164 are used to validate the successful decontamination of the interior of the aircraft. All personnel then exit the aircraft into the scissor jack trailer 106 where they are contained and decontaminated in a manner similar to that of the passengers (step 140c).

The hydrogen peroxide probes 160 (or other decontaminant detection probes) detect the level of decontaminant (hydrogen peroxide and/or water vapor in the illustrated embodiment) in the aircraft interior 100 to ensure that the hydrogen peroxide level in the interior 100 is maintained within a preselected range. The probes 160 are connected with a control system 166, which adjusts the rate of introduction of hydrogen peroxide to the vaporizer 145, air flow rates, or the like, in response to detected hydrogen peroxide/water concentrations, to maintain the selected hydrogen peroxide concentration in the aircraft interior 100 within the preselected range. Suitable hydrogen peroxide probes 160 are those which use infrared absorption by the vapor circulating in the aircraft interior 100. The probe preferably operates in a region of the infrared spectrum where water and/or hydrogen peroxide absorbs strongly, to provide a measure of the hydrogen peroxide concentration. Suitable aircraft internal air vents are opened and any vents or other potential sources of leakage to the atmosphere are closed or otherwise sealed.

Once the various probes 160 and indicators 162, 164 have been positioned and the aircraft is sealed, a decontamination cycle commenced (step 140d). A suitable decontamination cycle consists of four phases: dehumidification; conditioning; decontamination; and aeration. The entire cycle is controlled and monitored by the control system 166, which may be mounted on the truck 144. During the dehumidification phase, a dehumidifier 172 removes moisture and water vapor from the air, to increase the level of hydrogen peroxide which can be sustained in the environment without condensation. A heater 173 optionally heats the air to a suitable temperature. The air from the aircraft interior 100 is circulated to the dehumidifier and heater via the exit hose using a pump 174. Dehumidified air is returned to the aircraft interior 100 via the inlet hose. Optionally, a dehumidifier and blower in the aircraft's own air treatment system aids in dehumidifying the aircraft interior.

After reaching a designated relative humidity, the conditioning phase begins. In this phase, hydrogen peroxide vapor, generated by the vaporizer, is introduced to the aircraft interior 100 through the inlet port 146 via the inlet hose 150. The vapor is mixed with a carrier gas such as air, which is introduced from the exterior environment via an air inlet line 175 using a compressor or other suitable pump 176. Alternatively, all or part of the air is recirculated air from the aircraft interior. The air is directed to the vaporizer 146 via the dehumidifier 172 and/or heater 173. This air acts as a carrier gas to transport the vapor through the vaporizer and to the aircraft interior.

The aircraft interior eventually reaches a selected hydrogen peroxide concentration per liter of air. When that level is reached, the decontamination phase begins. The hydrogen peroxide vapor concentration is preferably maintained at a relatively constant level, by continually introducing the vapor into the incoming air and catalytically degrading the spent vapor in the returning air exiting the aircraft to water vapor and oxygen in a catalytic converter 177. The air exiting from the catalytic converter may be returned to the inlet line 175 (a recirculating system) or discharged to the atmosphere via a discharge line 178 (a non-recirculating system).

In one embodiment, the decontamination process can be considered a “dry” process, as the concentration is maintained close to but below the condensation point of the vapor. This avoids droplets of the vapor condensing on items in the aircraft interior 100, which both reduces the effectiveness of the vapor and increases the time needed to remove the residual hydrogen peroxide after the vapor decontamination cycle is complete. Keeping the vapor in the dry state also reduces the risk of damage to electronic components and other items susceptible to water damage.

The hydrogen peroxide vapor is flowed through the aircraft for a sufficient time to destroy microorganisms and chemical agents present in the air and within walls, ducts, and other structural parts of the aircraft, particularly the seats.

With reference to FIG. 3, the interior decontamination process optionally takes advantage of the existing air distribution system 180 of the aircraft. Specifically, the vapor is passed along air supply lines 182 to outlet vents 184 throughout the passenger compartment and cockpit. To supplement the existing circulation and dehumidification system 180, a blower 186 may be fitted between the air distribution system and the vents 184, or in a return flowpath, to increase the flowrate of the vapor throughout the aircraft interior 100. The air circulation and dehumidification system on board the aircraft may be used in place of or in combination with the dehumidifier 172 and pump 174 associated with the vaporizer to help dehumidify and circulate the air.

When the decontamination phase is complete, the injection of vapor is discontinued by the control system 166 and the aeration phase begins. Air for aeration may be fresh air, pumped in from line 175. The remaining hydrogen peroxide vapor passes out of the outlet hose 152 or is drawn out through the outlet hose by a pump 188 and passes through the catalytic converter 177, breaking it down to the environmentally acceptable byproducts of water vapor and oxygen, until the readout from the internal probes shows that an acceptable safe level of hydrogen peroxide is present in the aircraft interior 100. The remaining gas can be discharged to the atmosphere through an outlet line 178. Preferably the hydrogen peroxide concentration in the interior is reduced to about one part per million, which is the OSHA Permissible Exposure Limit (PEL), or less. In one embodiment, the hydrogen peroxide concentration in the aircraft interior 100 is reduced to about 0.5 ppm or less.

In one embodiment, the aeration proceeds for sufficient time using fresh air at or about ambient humidity to bring the hydrogen peroxide concentration in the aircraft interior 100 to a relatively low level (e.g., about 3-10 ppm). The humidity in the air is then reduced by operating the dehumidifier 172 and/or a dehumidifier 190 in the aircraft's circulation and dehumidification system 180. Aeration is continued with dehumidified air until the hydrogen peroxide reaches the acceptable safe level.

This two step aeration process has several advantages. Hydrogen peroxide tends to become absorbed by fabric and carpeting in the aircraft and is released relatively slowly. Dehumidification of the air speeds up the release and allows the aeration phase to be completed in a much shorter overall time. A level of one ppm or less can be readily achieved in about 4 hrs, or less. It is important that the dehumidification step is not carried out too early, i.e., before a relatively low level of hydrogen peroxide is achieved, as this can result in explosion of the hydrogen peroxide. Accordingly, the hydrogen peroxide concentration at which the dehumidification is commenced selected to be below that at which an explosion is likely to occur.

After completion of the decontamination cycle, SRT personnel enter the aircraft with the appropriate PPE, and collect the chemical indicators 164 and biological indicators 162. Concurrently, the inlet and outlet hoses 150, 152 are disconnected from the aircraft, contaminated equipment is loaded into tractor-trailers for decontamination, and the exteriors and interiors of all trucks and jet ways are decontaminated before leaving the site and being put back into service. The aircraft's air filters are removed and replaced. The exteriors of the vehicles are preferably decontaminated using the mobile decontaminant spraying truck 32, which sprays decontaminant over the exterior surfaces. Interiors can be decontaminated with the hydrogen peroxide generation system 142.

In one embodiment, the hoses 150, 152 from the vapor generation system 142 or separate hoses 210, 212 (FIG. 5) are connected with respective inlet and outlet ports 214, 216 on the passenger decontamination system 51 after all personnel have deported. Hydrogen peroxide vapor is circulated throughout the enclosure 55 for a sufficient time to ensure decontamination.

The chemical indicators 164 are analyzed for a positive change, assuring proper vapor dispersion. The biological indicators 162 are cultured, ideally indicating 100% eradication of pathogenic microorganisms. Once a successful confirmation of 100% eradication (or close to 100%) is confirmed on all the indicators, the aircraft is considered safe for personnel to enter without PPE. The aircraft can be restored to normal operations.

To destroy harmful biological pathogens in air and on surfaces throughout the aircraft interior 100, it has been found that a concentration of hydrogen peroxide of about 1-2 mg/L, or more at about 25° C. is effective to decontaminate the interior 100 in about 30 minutes or less. Longer times may be used at lower concentrations or for larger aircraft, or shorter times at higher concentrations or with small aircraft.

With reference to FIG. 2, the hydrogen peroxide vapor is readily formed from a solution of hydrogen peroxide in water, such as a 35% hydrogen peroxide solution, which is supplied from a reservoir 200, such as a tank, to the vaporizer 145. The vaporizer converts the liquid hydrogen peroxide and associated water to a vapor, for example, by bringing droplets or a mist of the solution into contact with a heated plate or tube (not shown). Other gaseous oxidizing agents may be used, such as peracids, e.g., peracetic acid vapor, ozone, or chlorine gas, alone, or in combination with one or more gaseous oxidants including hydrogen peroxide vapor.

The carrier gas, such as air, may be withdrawn from the aircraft, and/or may be fresh air drawn from the surrounding environment or supplied from a suitable tank (not shown). All or a portion of the air is passed along a fluid pathway 202. The air passes through the vaporizer along with the hydrogen peroxide solution. The carrier gas may be filtered by a filter 206, dehumidified by the dehumidifier 172, and optionally heated by the heater 173 before entering the vaporizer 145. The vapor and carrier gas mixture is fed into the inlet hose 150 and carried to the aircraft.

In one embodiment, a portion of the carrier gas bypasses the vaporizer, reaching the hose 150 by a bypass fluid line 208. This increases the throughput of the system.

Hazardous materials, which are removed from the aircraft or passengers are either incinerated or treated with a suitable decontaminant. Several different decontaminants are optionally utilized, depending on the type of material to be treated. For example, paper cloth, metal and plastic materials are optionally treated with ethylene oxide in a suitable sterilizer. Steam sterilization, vapor hydrogen peroxide, HEPA vacuuming, or the like may be appropriate for some materials.

For the system to be most effective, preplanning by airlines, airport management organizations, and national and state emergency response departments in the areas of detection, crisis consequence management and planning, systems integration, training, decontamination assistance and response, and post-incident humanitarian assistance is beneficial. Preplanning for the eventual release of contaminants will tend to reduce casualties, aid in the overall response effort, and eliminate the opportunity of further cross-contamination.

In preparing for an aircraft contamination event, personnel from these organizations are involved in planning, education awareness, training and drills, consequence management, and assessment of crisis capabilities. Additionally, Stand-Ready Teams are thoroughly trained on the entire end-to-end remediation and recovery decontamination process from the acceptance of the aircraft at the designated airport to the final disposition of the contaminated passengers, to the final confirmation that the aircraft is contamination free and safe to be put back into service.

The system is intended to remove or destroy all or substantially all contamination, preferably greater than 99%, more preferably greater than 99.99%) of all pathogenic microorganisms and pathogenic chemical agents. The hydrogen peroxide vapor treatment has been found to be effective against a wide variety of natural and man made or refined contaminants, such as chemical and biological warfare agents. Microorganisms which can be destroyed include bacteria (spore, vegetative, and mycobacteria), fungi, and viruses, and the toxins associated with such microorganisms.

Natural biological agents include SARS, Norwalk virus, anthrax, smallpox. These may be brought onto the aircraft by sick passengers or unintentionally carried on to the aircraft on the shoes, clothes or in the luggage of passengers who may not yet be infected. Biological warfare agents include biological microorganisms employed to disable personnel, as well as pesticides, herbicides, and similar substances which can be employed to interfere with the growth of plants, insects, and other non-mammalian species. They are commonly dispersed in aerosol form, as fine particles. For example, a passenger may have introduced the contaminant to the air using an aerosol container or by emptying a packet of small particles of contaminant which readily travel through the aircraft air circulation system. Explosive distribution activated by a remote system is also contemplated.

Included among the biological warfare agents are viruses, such as equine encephalomyelitis, Ebola, and smallpox (Variola); bacteria, such as those which cause plague (Yersina pestis), anthrax (B anthracis), brucellosis (e.g., Brucella melitensis, Brucella suis, Brucella abortus, and Brucella canis), and tularemia (Francisella tularensis); cholera (Vibrio cholerae), and fungi, such as Fusarium, Myrotecium and coccidioidomycosis; as well as toxic products expressed by such microorganisms; for example, the botulism toxin expressed by the common Clostridium botulinium bacterium, and ricin, a plant protein toxin derived from the beans of the castor plant. These microoganisms may have been refined, purified, or otherwise treated to increase their potency, such as in weapons grade anthrax.

Chemical warfare agents include poison gases and liquids, particularly those which are volatile, such as nerve gases, blistering agents (vesicants), and other extremely harmful or toxic chemicals. They are commonly dispersed as gases, smoke, or aerosols or by explosive means. As used herein, the term “chemical warfare agent” is intended to include only those agents which are effective in relatively small dosages to substantially disable or kill mammals. The term “chemical warfare agent” is not intended to encompass incendiaries, such as napalm, or explosives, such as gunpowder, TNT, nuclear devices, and so forth. Exemplary chemical warfare agents include choking agents, such as phosgene; blood agents, which act on the enzyme cytochrome oxidase, such as cyanogen chloride and hydrogen cyanide; incapacitating agents, such as 3-quinuclidinyl benzilate (“BZ”), vesicants, such as di(2-chloroethyl) sulfide (mustard gas or “HD”) and dichloro(2-chlorovinyl)arsine (Lewisite); nerve agents, such as ethyl-N, N dimethyl phosphoramino cyanidate (Tabun or agent GA), o-ethyl-S-(2-diisopropyl aminoethyl) methyl phosphono-thiolate (agent VX), isopropyl methyl phosphonofluoridate (Sarin or Agent GB), methylphosphonofluoridic acid 1,2,2-trimethylpropyl ester (Soman or Agent GD).

The hydrogen peroxide vapor reduces the activity of the microbial or chemical contaminant, either by killing a majority of the contaminant, as in the case of a microbial contaminant, or by converting the contaminant to a less harmful material, as in the case of a chemical contaminant. Large aircraft interiors and other enclosures can be decontaminated with the vapor, as well as items located in the aircraft interior 100, such as seats, carpeting, equipment, and the like.

It will be appreciated that while the invention has been described with particular reference to the decontamination of passenger carrying aircraft, it is also suited to the treatment of cargo-carrying aircraft and other large vehicles, such as cruise ships, commercial ships, trains, buses, and the like. Facilities where decontamination is found may also be treated, such as hospitals, schools, research facilities, postal facilities, and the like.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

1. A method for decontamination of an aircraft and contents thereof comprising:
removing people from the aircraft;
decontaminating the people with a liquid decontaminant capable of reducing the pathogenic activity of at least one of a biological agent and a chemical agent, where present, on skin of the people; and
decontaminating an interior of the aircraft with a peroxy vapor.

2. The method of claim 1, wherein the peroxy vapor includes hydrogen peroxide.

3. The method of claim 1, wherein the step of removing the people includes:
transferring the people to an enclosure which is isolated from the surrounding environment.

4. The method of claim 3, further including:
maintaining the enclosure at a negative pressure during the step of decontaminating the people.

5. The method of claim 3, wherein the step of decontaminating the people includes spraying the people within the enclosure with a decontamination solution.

6. The method of claim 3, wherein the step of transferring the people includes transferring the people through a jet way containment system which interconnects a door of the aircraft and the enclosure, the jet way containment system isolating the people from a surrounding environment.

7. The method of claim 3, wherein the enclosure is a mobile enclosure and the method further includes, prior to the step of removing the people from the aircraft:
transporting the mobile enclosure to a location adjacent the aircraft.

8. The method of claim 1, further including:
decontaminating an exterior of the aircraft with a decontaminant solution which is capable of at least one of destroying pathogenic microorganisms and reducing the activity of a pathogenic chemical agent.

9. The method of claim 1, wherein the step of decontaminating the aircraft interior includes:
delivering hydrogen peroxide from a vaporizer to a port on the aircraft, the port being fluidly connected with an air circulation system of the aircraft; and
circulating the hydrogen peroxide vapor to vents in the aircraft interior with the air circulation system.

10. The method of claim 1, wherein the step of decontamination of the aircraft interior includes:
blowing the peroxy vapor into the interior of the aircraft with a blower.

11. The method of claim 10, further including:
connecting the blower with air circulation system of the aircraft, the blower increasing a rate of flow of the air circulation system.

12. The method of claim 2, further including:
sensing a level of hydrogen peroxide in the aircraft interior and controlling the rate of hydrogen peroxide vapor introduction to the interior to maintain a preselected hydrogen peroxide concentration within the interior.

13. The method of claim 1, further including:
after the step of decontaminating the aircraft interior, aerating the aircraft interior to remove residual peroxy vapor.

14. The method of claim 13, further including:
after the aeration has reduced the peroxy vapor to a level which is below that at which explosion is likely to occur, dehumidifying air entering the interior.

15. The method of claim 1, further including:
removing luggage from the aircraft; and
decontaminating the luggage.

16. A system for decontamination of a passenger aircraft and people on the aircraft comprising:
an enclosure which receives people from the aircraft;
a means for decontaminating the people associated with the enclosure; and
a decontamination system configured for selective connection with the aircraft for decontamination of an interior of the aircraft.

17. The system of claim 16, wherein the enclosure is carried by a vehicle for moving the enclosure to a location adjacent an aircraft passenger exit door.

18. The system of claim 16, wherein the means for decontaminating includes a shower head which sprays a microbial decontaminant solution over the people.

19. The system of claim 18, wherein the microbial decontaminant includes peracetic acid.

20. The system of claim 16, wherein the enclosure includes means for maintaining the enclosure at a negative pressure.

21. The system of claim 16, further including a jet way which selectively connects a door of the aircraft with the enclosure, the jet way isolating people from a surrounding environment during transfer from the aircraft to the enclosure.

22. The system of claim 16, wherein the means for decontaminating the people includes:
a source of a decontamination solution which is capable of at least one of destroying pathogenic microorganisms and reducing the activity of a chemical pathogenic agent; and
a shower head located within the enclosure.

23. A system for decontamination of passengers from a vehicle or from within a facility who have been exposed to at least one of pathogenic biological agents and chemical agents comprising:
a mobile enclosure capable of being coupled to an exit door of the vehicle for receiving passengers from the vehicle or facility, the enclosure spacing the received passengers from the surrounding environment; and
a decontaminant delivery system which supplies a decontaminant to the enclosure, the decontaminant being capable of destroying the pathogenic biological or chemical agent, the delivery system including at least one liquid outlet within the enclosure which sprays the decontaminant on the passengers.

24. The system of claim 23, further including:
means for maintaining a negative pressure within the enclosure.

25. The system of claim 23, further including:
a jet way containment system which selectively couples the enclosure with the exit door, the jet way containment system spacing the passengers from the surrounding environment during transfer of the passengers from the vehicle or facility to the enclosure.
